(19) United States
(12) Patent Application Publication
(10) Pub. No.: US 2011/0201027 A1
(43) Pub. Date: Aug. 18, 2011

(54) BIOSENSOR FOR DETECTING A TRACE AMOUNT OF SAMPLE AND PRODUCTION METHOD THEREFOR

(75) Inventors: Min Gon Kim, Daejeon (KR); Yong Beom Shin, Daejeon (KR); Bong Hyun Chung, Daejeon (KR); Jun Hyoung Ahn, Busan (KR)

(73) Assignee: KOREA RESEARCH INSTITUTE OF BIOSCIENCE AND BIOTECHNOLOGY, Daejeon (KR)

(21) Appl. No.: 12/933,114

(22) PCT Filed: Mar. 18, 2009
(86) PCT No.: PCT/KR2009/001374

§ 371 Date: Oct. 28, 2010

(30) Foreign Application Priority Data
Mar. 18, 2008 (KR) 10-2008-0024796

(51) Int. Cl. G01N 33/53; C12M 1/34; G01N 1/28

(52) U.S. Cl. 435/7.21; 435/7.8; 435/7.4; 435/7.1; 435/287.9; 427/2.13

(57) ABSTRACT

The present invention relates to a biosensor capable of a trace amount of sample and a fabrication method thereof. More specifically, the invention relates to a method for fabricating a biosensor, the method comprising: immobilizing a receptor molecule, which binds selectively to a target substance, on an electrically insulated nano-electrode chip; binding an enzyme to the receptor molecule; and treating the bound enzyme with metal ions and depositing the metal ions on the nano-electrode surface, and to a biosensor fabricated thereby. According to the invention, a precipitate is produced on the nano-electrode surface by a precipitation between the enzyme and the metal ions, and the produced precipitate electrically connects the nano-electrodes together, thereby increasing electrical conductivity. Thus, the invention is useful for quantitative analysis of trace amounts and/or various concentrations of target substances.

### FIELD OF INVENTION

The present invention relates to a biosensor capable of selectively measuring a trace amount of sample and a manufacture method thereof, and more particularly to a method for fabricating a biosensor, the method comprising: immobilizing a receptor molecule, which binds selectively to a target substance, on an electrically insulated nano-electrode chip; binding an enzyme to the receptor molecule; and treating the bound enzyme with metal ions and depositing the metal ions on the nano-electrode surface, and to a biosensor fabricated thereby.

### BACKGROUND ART

Technology for quantitatively measuring trace amounts of biomolecules such as protein or DNA is important in clinical diagnosis, proteomics studies and the like.

In the prior art, an ELISA (enzyme-linked immunosorbent assay) method was mainly used to measure proteins. The ELISA method comprises: immobilizing an antibody on a matrix; allowing an antigen protein-containing sample solution to react with the antibody-immobilized surface for a given time; binding a detecting antibody, which binds selectively to the antigen protein, to the antigen protein-bound surface; binding an enzyme to the detecting antibody; and adding a reaction material, enabling the enzyme to cause color development or fluorescence characteristics so as to cause a color development or fluorescence reaction, to the detecting antibody, thereby quantifying a trace amount of the antigen protein. Although the ELISA method has been used to date as a method for effectively measuring trace amounts of antigen proteins, it can analyze antigen proteins in an amount ranging from about 10 pg/ml to 1 ng/ml and can analyze only one antigen protein a time. For this reason, the ELISA method has a disadvantage in that it requires a large amount of sample in order to analyze a variety of antigen proteins.

Recently, the principle of ELISA has been applied to biosensors and biochips in order to develop various methods of measuring the concentration of antigen in a simple and easy manner. For example, methods utilizing the principle of ELISA have been reported in which an enzyme attached to a surface in proportion to the concentration of antigen induces a precipitation reaction such that the enzyme reaction product is attached to the surface, and then measurements are carried out by surface plasmon resonance (SPR), quartz crystal microbalance (QCM), electrochemical sensors, etc. Such methods can measure trace amounts of antigens in a simple and easy manner and can also simultaneously measure a variety of antigens (Kim et al., J. Immunol. Meth., 297:125, 2005; Abad et al., Anal. Chim. Acta, 368:183, 1998). However, such methods do not show a great improvement in measurement sensitivity compared to the existing ELISA method.

Accordingly, the present inventors have made many efforts to develop a biosensor capable of measuring a trace amount of sample and, as a result, have found that, when a biosensor manufactured by connecting nano-electrodes to each other through an enzyme-metal ion precipitation reaction is used to measure a target substance, the electrical conductivity between the nano-electrodes can be increased such that a trace amount of the target substance can be easily measured, thereby completing the present invention.

### DISCLOSURE OF INVENTION

It is a main object of the present invention to provide a biosensor capable of measuring a trace amount of sample and a fabrication method thereof.

Another object of the present invention is to provide a method of detecting a target substance using said biosensor.

To achieve the above objects, the present invention provides a method for fabricating a biosensor, the method comprising the steps of: (a) immobilizing a receptor molecule, which binds selectively to a target substance to be analyzed, on an electrically insulated nano-electrode chip; (b) binding an enzyme to the immobilized receptor in direct or inverse proportion to the concentration of the target substance; and (c) treating the bound enzyme with metal ions, depositing the metal ions on the nano-electrode surface by an enzyme precipitation reaction, and then washing and drying the nano-electrode.

The present invention also provides a biosensor which is fabricated according to said method and in which metal ions are deposited on the surface of nano-electrodes.

The present invention also provides a method for detecting a target substance, the method comprising the steps of: (a) applying a sample containing the target substance to said biosensor; and (b) measuring electrical conductivity to detect the target substance binding specifically to a receptor on the biosensor.

Other features and embodiments of the present invention will be more fully apparent from the following detailed description and appended claims.

### BRIEF DESCRIPTION OF THE DRAWINGS

FIG. 1 is a schematic diagram showing a method of quantitatively analyzing a trace amount of target substance using a biosensor comprising nano-electrode biosensors connected together by an enzyme-metal ion precipitation reaction. In FIG. 1, A: a schematic diagram showing the nano-electrode biosensor; 1: nano-sized space; 11: metal electrode; 21: insulator surface; B: a schematic diagram showing a method of quantitatively analyzing a target substance using a biosensor comprising nano-electrode biosensors connected together by an enzyme-metal ion precipitation reaction; 31: enzyme; and 41: precipitate resulting from enzyme reaction.

FIG. 2 shows a photographic image of a substrate according to one embodiment of the present invention.

FIG. 3 shows an atomic force microscope (AFM) image of a substrate according to one embodiment of the present invention.

FIG. 4 shows an SEM image of nano-electrodes fabricated by an FIB process.

FIG. 5 shows the results of measuring current 10 minutes after reacting each of an enzyme-immobilized nano-electrode and an enzyme-nonimmobilized nano-electrode with a substrate (FIG. 5A; red: enzyme-immobilized nano-electrode; blue: enzyme-nonimmobilized nano-electrode) and the results of measuring changes in current that flowed at a voltage of 1V for 30 minutes, as a function of time (FIG. 5B).

FIG. 6 shows an atomic force microscope (AFM) image of a substrate according to one embodiment of the present invention.

FIG. 7 shows an SEM image of nano-electrodes fabricated by an FIB process.

FIGS. 8 to 10 show the result after reacting each of an enzyme-immobilized nano-electrode and an enzyme-nonimmobilized nano-electrode with a substrate.

FIG. 8 shows the results of measuring current 30 minutes after reacting each of an enzyme-immobilized nano-electrode and an enzyme-nonimmobilized nano-electrode with a substrate.

FIG. 9 shows the results of measuring changes in current that flowed at a voltage of 1 V for 60 minutes, as a function of time.

FIG. 10 shows the results of measuring current 5 minutes after the start of an enzyme reaction at various enzyme concentrations.

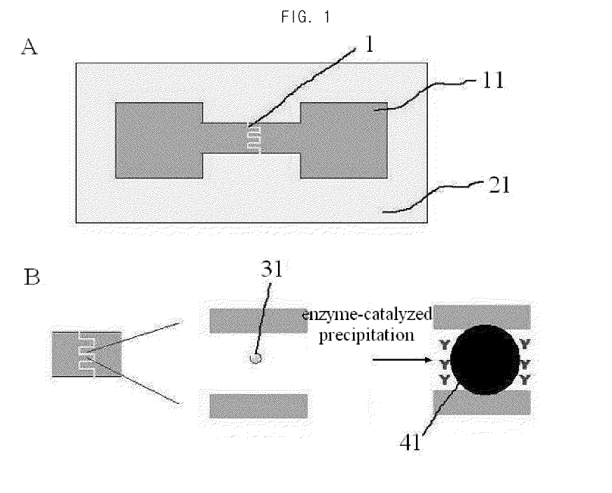

### DETAILED DESCRIPTION OF THE INVENTION AND BEST MODE FOR CARRYING OUT THE INVENTION

In one aspect, the present invention is directed to a biosensor capable of measuring a trace amount of sample and a fabrication method thereof.

The biosensor according to the present invention can be fabricated by the following method comprising the steps: (a) immobilizing a receptor molecule, which binds selectively to a target substance to be analyzed, on an electrically insulated nano-electrode chip; (b) binding an enzyme to the immobilized receptor in direct or inverse proportion to the concentration of the target substance; and (c) treating the bound enzyme with metal ions, depositing the metal ions on the nano-electrode surface by an enzyme precipitation reaction, and then washing and drying the nano-electrode.

In the present invention, the nano-electrode chip is preferably fabricated by a process selected from the group consisting of photolithography, electron beam lithography, focused ion beam lithography, and nanoimprinting. The nano-electrode chip preferably has an insulation distance of less than 1 μm. When an increase in current by an enzyme precipitation reaction on a micro-electrode of more than 1 μM is measured, a large amount of enzyme should be bound or a separate reaction following the enzyme precipitation reaction should be induced, but in the case of a nano-electrode of less than 1 μM, an increase in electrical conductivity can be induced by a one-step enzyme precipitation reaction.

In the present invention, the target substance is preferably selected from the group consisting of enzymes, proteins, DNA, RNA, microorganisms, animal and plant cells and organs and nerve cells. The receptor molecule is preferably selected from the group consisting of antibodies, DNA, aptamers, PNAs (peptide nucleic acids) and ligands.

In the present invention, the enzyme is preferably peroxidase or phosphatase, and the metal ions are preferably selected from the group consisting of gold, silver and copper ions.

In another aspect, the present invention is directed to a method of detecting a target substance using said biosensor.

Many studies on a method of electrically connecting micro-electrodes with each other by forming precipitates through enzyme reactions are in progress (Moller et al., Nano Lett., 5:1475, 2005; WO 02/103037). However, such studies have shortcomings in that, because the micro-electrodes are used, the amount of enzyme binding to the electrode surface should be large or an additional precipitation reaction following the enzyme precipitation reaction should be induced, in order to electrically connect the electrodes together such that current flows between the electrodes. In comparison with this, according to the present invention, a precipitate formed on nano-electrodes by a one-step enzyme precipitation reaction electrically connects the insulated nano-electrodes together, such that when a given voltage is applied between the electrodes, current will flow between the electrodes. Accordingly, the nano-electrodes of the present invention have an advantage in that they can be used to quantitatively measure a target substance.

Meanwhile, a method of measuring a target substance using nano-electrodes having gold nanoparticles bound thereto was reported (Malaquin et al., Microelectron. Eng., 73˜74:887, 2004). This method has a shortcoming in that, because the gold nanoparticles do not completely connect the electrodes together, an increase in current is not significant. On the other hand, the present invention has advantages in that, because nano-electrodes having an enzyme bound thereto are used, one enzyme molecule connects the electrodes together through its precipitation reaction, such that the intensity of current can be increased, whereby analysis sensitivity can be increased.

In one Example of the present invention, the surface of a glass substrate was modified with an amine group, and the amine-modified substrate surface was modified with a biotin group. The biotin-modified substrate surface was immobilized with streptavidin-alkaline phosphatase, and then treated with indoxyl phosphatase disodium salt and silver nitrate to induce an enzyme precipitation reaction so as to produce a precipitate on the substrate surface. Then, the substrate was washed and dried with nitrogen. Formation of a precipitate on the substrate surface was observed with an atomic force microscope (AFM).

In another Example of the present invention, a self-assembled monomolecular film was formed on the surface of a nano-electrode fabricated using an FIB process, followed by washing and drying. The dried nano-electrode surface was modified with an amine group, and the amine-modified substrate surface was modified with a biotin group. The biotin-modified nano-electrode surface was immobilized with streptavidin-alkaline phosphatase, and then treated with indoxyl phosphatase disodium salt and silver nitrate to induce an enzyme precipitation reaction so as to produce a precipitate on the substrate surface. Then, the substrate was washed and dried with nitrogen.

Each of a nano-electrode not immobilized with enzyme and the above-prepared enzyme-immobilized electrode was allowed to react with a substrate, and current was allowed to flow through each electrode. Then, the measurement of current was carried out. As a result, the enzyme-immobilized nano-electrode showed increased electrical conductivity compared to the enzyme-nonimmobilized nano-electrode.

In still another Example of the present invention, the surface of a glass substrate was modified with an amine group, and the amine-modified substrate surface was modified with a biotin group. The biotin-modified substrate surface was immobilized with streptavidin-peroxidase, and then treated with silver acetate, glutathione, hydroquinone and hydrogen peroxide to induce an enzyme precipitation reaction so as to produce a precipitate on the substrate surface. Then, the substrate was washed and dried with nitrogen gas. Formation of the precipitate on the substrate surface was observed with an AFM.

In yet another Example of the present invention, a self-assembled monocular film was formed on the surface of a nano-electrode fabricated using an FIB process, followed by washing and drying. The dried nano-electrode surface was modified with an amine group, and the amine-modified substrate surface was modified with a biotin group. The biotin-modified nano-electrode surface was immobilized with streptavidin-peroxidase, and then treated with silver acetate, glutathione, hydroquinone and hydrogen peroxide to induce an enzyme precipitation reaction so as to produce a precipitate on the substrate surface. Then, the substrate was washed and dried with nitrogen gas. Each of a nano-electrode not immobilized with enzyme and the above-prepared enzyme-immobilized electrode was allowed to react with a substrate, and current was allowed to flow through each electrode. Then, the measurement of current was carried out. As a result, the enzyme-immobilized nano-electrode showed increased electrical conductivity compared to the enzyme-nonimmobilized nano-electrode.

Accordingly, in the present invention, the concentration of a target substance to be analyzed can be measured by linking an enzyme in direct or inverse proportion to various binding reactions, including antigen-antibody, DNA-DNA, DNA-RNA, PNA-DNA, etc., and then inducing an enzyme-metal ion precipitation reaction, followed by measuring current or resistance.

### EXAMPLES

It will be obvious to a person having ordinary skill in the art that these embodiments are merely for illustrative purposes, and the scope of the present invention should not be construed as being limited to the above described embodiments.

### Example 1

### Silver Precipitation Reaction of Alkaline Phosphatase Immobilized on Substrate

A glass substrate (18×18 mm) was immersed in a mixed solution of 95% sulfuric acid and 30% hydrogen peroxide (3:1 v/v) at 60-65° C. for 20 minutes, and then washed sequentially with distilled water and ethanol. The washed glass substrate was immersed in a 1% ethanol solution of 3-aminopropyltrimethoxysilane (Aldrich, St. Louis, Mo., USA) for 1 hour, washed with ethanol and maintained at 100° C. for 1 hour, thereby modifying the substrate surface with an amine group.

In order to modify the amine-modified substrate surface with a biotin group, NHS-biotin (Sigma, St. Louis, Mo., USA) was dissolved in dimethyl sulfoxide (DMSO, Simga-Aldrich, St. Louis, Mo., USA) at a concentration of 10 mg/ml, and then added to PBS buffer at a concentration of 1 mg/ml, thus preparing a solution. Then, the substrate surface was allowed to react with the above-prepared solution at room temperature for 1 hour, thereby substituting the amine group with the biotin group. The biotin-modified substrate surface was treated with a solution of 0.1 mg/ml of streptavidin-alkaline phosphatase (STA-AP, Sigma, St. Louis, Mo., USA) in 0.1M PB buffer (pH 7.0), and then allowed to react with the solution at room temperature for 1 hour, thereby immobilizing the enzyme on the glass substrate surface.

Subsequently, the enzyme-immobilized surface was treated with a solution of 1 mM indoxyl phosphate disodium salt (Sigma, St. Louis, Mo., USA) and 1 mM silver nitrate (Sigma-Aldrich, St. Louis, Mo., USA) in 0.1M Tris buffer (pH 9.8) and allowed to react with the solution at room temperature for 1 hour so as to produce a precipitate on the substrate surface. Then, the substrate was washed and dried with nitrogen gas.

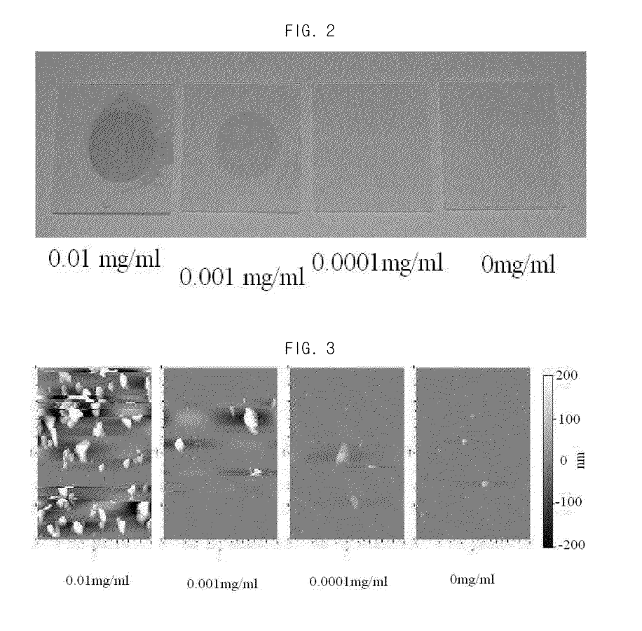

FIG. 2 shows a photographic image of the above-fabricated glass substrate. As can be seen therein, when the concentration of streptavidin-alkaline phosphatase was 0.1 mg/mL or 0.01 mg/mL, a color appeared due to the formation of silver nanoparticles.

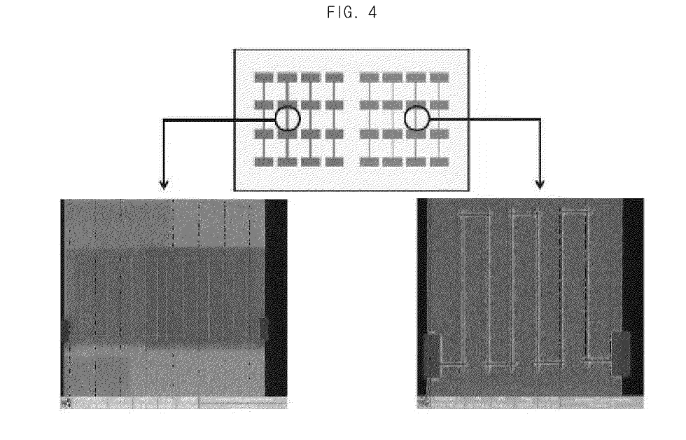

FIG. 3 shows an atomic force microscope (AFM) image of the above substrate. As can be seen therein, as the concentration of streptavidin-alkaline phosphatase increased, the formation of silver nanoparticles of more than 200 nm was increased.

### Example 2

### Precipitation Reaction of Alkaline Phosphatase on Nano-Electrodes

A gold nano-electrode was fabricated using an FIB process (FIG. 4). Namely, Si₃N₄ was deposited on a silicon wafer by chemical vapor deposition to form a thin film. Then, the wafer surface was patterned by photolithography so as to form lines having a line width of 20 μm, and 5 nm chromium (Cr)/30 nm gold (Au) was deposited on the surface by a thermal evaporator, followed by fabricating electrodes using a lift-off process. The electrodes having the metal lines of 20 μm were etched into a specific shape using a focused ion beam (FIB). Herein, an ion beam voltage of 30 kV and an ion beam current of 50 pA were used. FIG. 4 shows an SEM image of the nano-electrodes fabricated using the above process. As can be seen therein, nano-electrodes insulated at a distance of about 106 nm were fabricated.

The above-fabricated nano-electrode was immersed in a mixed solution of 95% sulfuric acid and 30% hydrogen peroxide (3:1 v/v) at 60-65° C. for 20 minutes, and then washed sequentially with distilled water and ethanol. The washed nano-electrode was immersed in a solution of 20 mM HS-(PEG)6-OH (Cosbiotech, Daejeon, Korea) and 20 mM 2-mercaptoethanol (Sigma, St. Louis, Mo., USA) in ethanol at room temperature for about 12 hours to form a self-assembled monomolecular film on the surface. Then, the nano-electrode was washed sequentially with ethanol and distilled water and dried with nitrogen gas. The dried nano-electrode was immersed in an 1% ethanol solution of 3-aminopropyltrimethoxysilane (Aldrich, St. Louis, Mo., USA) for 1 hour, and then the electrode surface was washed with ethanol and dried with nitrogen gas. The dried nano-electrode was maintained at 100° C. for 1 hour, thereby modifying the silicon surface of the gold nano-electrode with an amine group.

In order to modify the amine-modified surface with a biotin group, NHS-biotin (Sigma, St. Louis, Mo., USA) was dissolved in dimethyl sulfoxide (DMSO, Simga-Aldrich, St. Louis, Mo., USA) at a concentration of 10 mg/ml, and then added to PBS buffer at a concentration of 1 mg/ml, thus preparing a solution. Then, the surface was allowed to react with the above-prepared solution at room temperature for 1 hour, thereby substituting the amine group with a biotin group. The biotin-modified surface was treated with a solution of 0.1 mg/ml of streptavidin-alkaline phosphatase (STA-AP, Sigma, St. Louis, Mo., USA) in 0.1M PB buffer (pH 7.0), and then allowed to react with the solution at room temperature for 1 hour, thereby immobilizing the enzyme on the silicon surface between the gold electrodes.

Subsequently, the enzyme-immobilized surface was treated with a solution of 0.5 mM indoxyl phosphate disodium salt (Sigma, St. Louis, Mo., USA) and 0.1 mM silver nitrate (Sigma-Aldrich, St. Louis, Mo., USA) in 0.1M tris-HNO₃ buffer (pH 9.8) and allowed to react with the solution at room temperature for 1 hour so as to produce a precipitate on the enzyme-immobilized surface. Then, the nano-electrode was washed with distilled water and dried with nitrogen gas.

Each of an enzyme-nonimmobilized nano-electrode and the above-fabricated enzyme-immobilized nano-electrode was allowed to react with a substrate, and current was allowed to flow through each electrode. After 10 minutes, the measurement of current was carried out. Also, changes in the current that flowed at a voltage of 1V for 30 minutes were measured as a function of time.

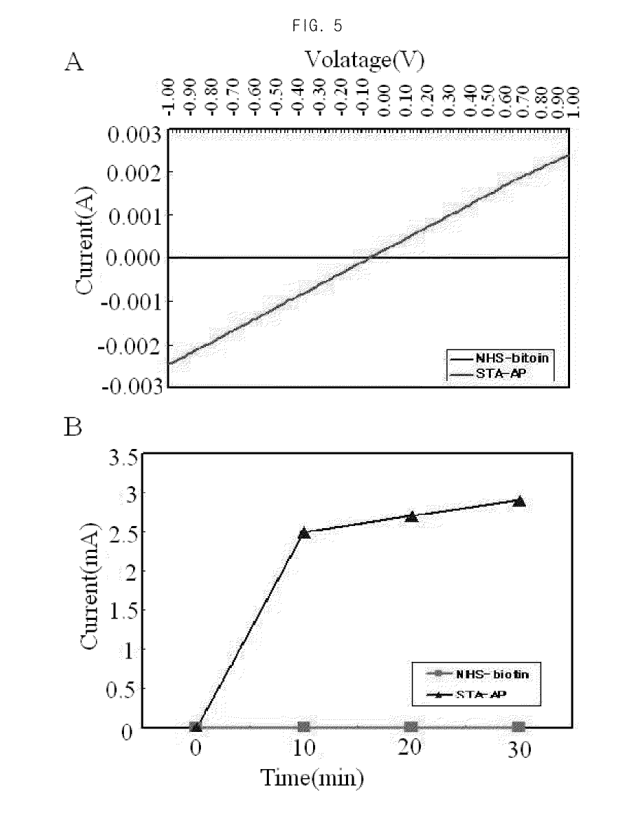

As a result, it could be seen that, in the case of the enzyme-nonimmobilized nano-electrode, when a voltage of 1 V was applied, a current of 1 μA flowed, whereas in the case of the enzyme-immobilized nano-electrode, a current of 2.5 mA flowed (FIG. 5). This suggests that the enzyme-immobilized nano-electrode induces an increase in electrical conductivity compared to the enzyme-nonimmobilized nano-electrode.

### Example 3

### Silver Precipitation Reaction of Peroxidase Immobilized on Substrate

A glass substrate (18×18 mm) was immersed in a mixed solution of 95% sulfuric acid and 30% hydrogen peroxide (3:1 v/v) at 60-65° C. for 20 minutes, and then washed sequentially with distilled water and ethanol. The washed glass substrate was immersed in a 1% ethanol solution of 3-aminopropyltrimethoxysilane (Aldrich, St. Louis, Mo., USA) for 1 hour, and then washed with ethanol. The washed substrate was maintained at 100° C. for 1 hour, thereby modifying the substrate surface with an amine group.

In order to modify the amine-modified surface with a biotin group, NHS-biotin (Sigma, St. Louis, Mo., USA) was dissolved in dimethyl sulfoxide (DMSO, Simga-Aldrich, St. Louis, Mo., USA) at a concentration of 10 mg/ml, and then added to PBS buffer at a concentration of 1 mg/ml, thus preparing a solution. Then, the surface was allowed to react with the above-prepared solution at room temperature for 1 hour, thereby substituting the amine group with a biotin group. The biotin-modified substrate surface was treated with a solution of 0.1 mg/ml of streptavidin-alkaline phosphatase (STA-AP, Sigma, St. Louis, Mo., USA) in 0.1M PB buffer (pH 7.0), and then allowed to react with the solution at room temperature for 1 hour, thereby immobilizing the enzyme on the glass substrate.

Subsequently, the enzyme-immobilized surface was treated with a solution of 1 mM silver acetate (Sigma, St. Louis, Mo., USA), 10 mM hydroquinone (Oriental Chemical Industries, Seoul, Korea), 1 mM reduced glutathione (Duchefa Biochemie, Haarlem, Netherlands) and 1 mM hydrogen peroxide (Junsei, Tokyo, Japan) in 0.1M citrate buffer (pH 8.5) and allowed to react with the solution at room temperature for 1 hour so as to produce a precipitate on the surface. The substrate was washed with distilled water and dried with nitrogen gas.

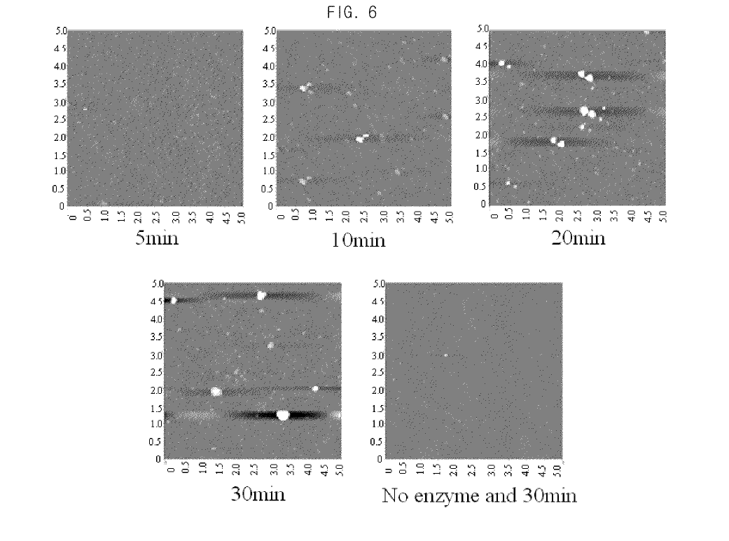

FIG. 6 shows an atomic force microscope (AFM) image of the above-fabricated glass substrate. As can be seen therein, the reaction time of streptavidin-alkaline phosphatase increased, the formation of silver nanoparticles of more than 200 nm was increased.

### Example 4

### Precipitation Reaction of Peroxidase on Nano-Electrodes

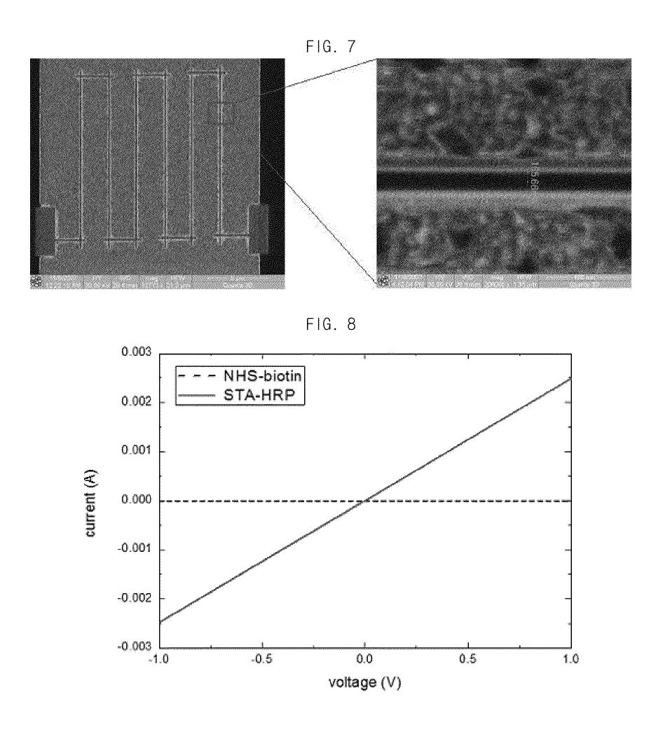

Gold nano-electrodes were fabricated using an FIB process (FIG. 7). For this purpose, Si₃N₄ was deposited on a silicon wafer by chemical vapor deposition to form a thin film. The surface was patterned by photolithography to form lines having a line width of 20 μm, and 5 nm Cr/30 nm Au was deposited on the surface by a thermal evaporator. Electrodes were formed on the surface using a lift-off process. The electrodes having the metal lines of 20 μm were etched into a specific shape using a focused ion beam (FIB). Herein, an ion beam voltage of about 30 kV and an ion beam current of about 50 pA were used. FIG. 7 shows an SEM image of the nano-electrodes fabricated using the above-described process. As can be seen therein, insulated nano-electrodes were fabricated at a distance of about 106 nm.

The above-fabricated nano-electrode was immersed in a mixed solution of 95% sulfuric acid and 30% hydrogen peroxide (3:1 v/v) at 60-65° C. for 20 minutes, and then washed sequentially with distilled water and ethanol. The washed electrode was immersed in a solution of 20 mM HS-(PEG)6-OH (Cosbiotech, Daejeon, Korea) and 20 mM 2-mercaptoethanol (Sigma, St. Louis, Mo., USA) in ethanol at room temperature for about 12 hours so as to form a self-assembled monomolecular film on the surface. Then, the electrode was washed sequentially with ethanol and distilled water and dried with nitrogen gas. The dried nano-electrode was immersed in a 1% ethanol solution of 3-aminopropyltrimethoxysilane (Aldrich, St. Louis, Mo., USA) in ethanol for 1 hour, and then the electrode surface was washed with ethanol and dried with nitrogen gas. Then, the electrode was maintained at 100° C. for 1 hour, thereby modifying the silicon surface of the gold nano-electrode with an amine group.

In order to modify the amine-modified surface with a biotin group, NHS-biotin (Sigma, St. Louis, Mo., USA) was dissolved in dimethyl sulfoxide (DMSO, Simga-Aldrich, St. Louis, Mo., USA) at a concentration of 10 mg/ml, and then added to PBS buffer at a concentration of 1 mg/ml, thus preparing a solution. Then, the surface was allowed to react with the above-prepared solution at room temperature for 1 hour, thereby substituting the amine group with a biotin group. The biotin-modified substrate surface was treated with a solution of 0.1 mg/ml of streptavidin-alkaline phosphatase (STA-AP, Sigma, St. Louis, Mo., USA) in 0.1M PB buffer (pH 7.0), and then allowed to react with the solution at room temperature for 1 hour, thereby immobilizing the enzyme on the silicon surface between the gold electrodes.

Subsequently, the enzyme-immobilized surface was treated with a solution of 1 mM silver acetate (Sigma, St. Louis, Mo., USA), 10 mM hydroquinone (Oriental Chemical Industries, Seoul, Korea), 1 mM reduced glutathione (Duchefa Biochemie, Haarlem, Netherlands) and 1 mM hydrogen peroxide, Junsei, Tokyo, Japan) in 0.1M citrate buffer (pH 8.5) and allowed to react with the solution at room temperature for 1 hour so as to produce a precipitate on the enzyme-immobilized surface. Then, the nano-electrode was washed with distilled water and dried with nitrogen gas.

Each of an enzyme-nonimmobilized nano-electrode and the above-fabricated enzyme-immobilized nano-electrode was allowed to react with a substrate, and current was allowed to flow through each electrode. After 10 minutes, the measurement of current was carried out. Also, changes in the current that flowed at a voltage of 1V for 30 minutes were measured as a function of time.

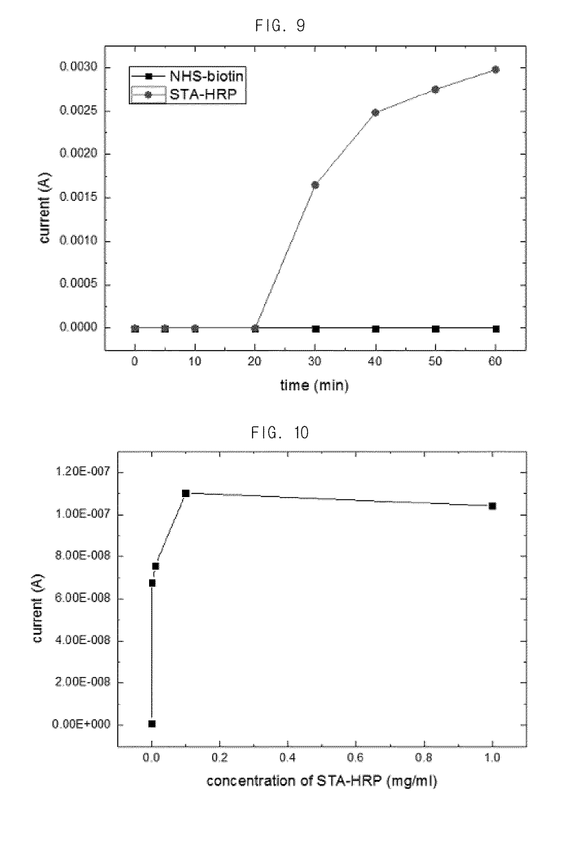

As a result, it could be seen that, in the case of the enzyme-nonimmobilized nano-electrode, when a voltage of 1 V was applied, a current of 1 μA flowed, whereas in the case of the enzyme-immobilized nano-electrode, a current of 2.5 mA flowed (FIG. 8). FIG. 9 shows changes in current at a voltage of 1 V as a function of enzyme reaction time, and FIG. 10 shows the results of measuring current 5 minutes after the start of enzyme reaction as a function of enzyme concentration. As can be seen from the results of FIGS. 8 to 10, the enzyme-immobilized nano-electrode induced an increase in electrical conductivity compared to the enzyme-nonimmobilized nano-electrode.

### INDUSTRIAL APPLICABILITY

As described in detail above, when the biosensor according to the present invention is used to measure a target substance to be analyzed, the nano-electrodes can be connected together by a one-step enzyme-metal ion precipitation reaction, and the electrically insulated nano-electrodes can also be electrically connected together to increase electrical conductivity. Accordingly, the biosensor according to the present invention has high measurement sensitivity, can measure a trace amount of sample, and can also electrically measure selective antigen-antibody binding in a very simple manner.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for fabricating a biosensor, the method comprising the steps of: (a) immobilizing a receptor molecule, which binds selectively to a target substance to be analyzed, on an electrically insulated nano-electrode chip; (b) binding an enzyme to the immobilized receptor in direct or inverse proportion to the concentration of the target substance; and (c) treating the bound enzyme with metal ions, depositing the metal ions on the nano-electrode surface by an enzyme precipitation reaction, and then washing and drying the nano-electrode.

2. The method according to claim 1, the nano-electrode chip is fabricated by a process selected from the group consisting of photolithography, electron beam lithography, focused ion beam lithography, and nanoimprinting.

3. The method according to claim 1, the nano-electrode chip has an insulation distance of less than 1 μm.

4. The method according to claim 1, the target substance is selected from the group consisting of enzymes, proteins, DNA, RNA, microorganisms, animal cells, plant cells, organs and nerve cells.

5. The method according to claim 1, the receptor molecule is selected from the group consisting of antibodies, DNA, aptamers, PNAs (peptide nucleic acids) and ligands.

6. The method according to claim 1, the enzyme is peroxidase or phosphatase.

7. The method according to claim 1, the metal ions are selected from the group consisting of gold, silver and copper ions.

8. A biosensor which is fabricated according to the method of claim 1 and in which metal ions are deposited on the surface of nano-electrodes.

9. A method for detecting a target substance, the method comprising the steps of: (a) applying a sample containing the target substance to the biosensor according to claim 8; and (b) measuring electrical conductivity to detect the target substance binding specifically to a receptor on the biosensor.

10. The method according to claim 9, the target substance is selected from the group consisting of enzymes, proteins, DNA, RNA, microorganisms, animal cells, plant cells, organs and nerve cells.
